(19) United States
(12) Patent Application Publication
(10) Pub. No.: US 2023/0330291 A1
(43) Pub. Date: Oct. 19, 2023

(54) CHEMICAL SPRAYING SYSTEM AND METHOD FOR OPERATING SAME

(71) Applicant: Panasonic Intellectual Property Management Co., Ltd., Osaka (JP)

(72) Inventors: Kota KIMURA, Saitama (JP); Takeshi OGATA, Tokyo (JP)

(21) Appl. No.: 18/019,263

(22) PCT Filed: Oct. 11, 2021
(86) PCT No.: PCT/JP2021/037607

§ 371 Date: Feb. 2, 2023

(30) Foreign Application Priority Data
Oct. 13, 2020 (JP) 2020-172678

(51) Int. Cl. A61L 2/24; B05B 12/12; A61L 2/00

(52) U.S. Cl. CPC A61L 2/24; B05B 12/122; A61L 2/0088; A61L 2202/14; A61L 2202/15

(57) ABSTRACT

A control unit performs control such that the mist is started to be sprayed in a horizontal direction or a downward direction from a spraying unit toward a subject on a basis of detection of the subject by a detection unit.

### TECHNICAL FIELD

The present disclosure relates to a chemical spraying system that sprays a sanitizing solution and water in mist form for sanitization or sterilization, and a method for operating the same.

### BACKGROUND ART

A system for spraying a sanitizing solution is used for sanitization or sterilization. For example, PTL 1 discloses a sanitizing apparatus that supplies water containing a sanitizing component into a room to perform sanitization.
PTL 1: JP 2012-075487 A

### SUMMARY OF INVENTION

In the conventional sanitizing apparatus as described above, for example, psychological anxiety of a user such as inhaling a sanitizing solution in a case where the sanitizing solution is sprayed on the human body has not been examined.

Therefore, an object of the chemical spraying system and the method for operating the same of the present disclosure is to be able to perform spraying while reducing psychological anxiety of the user.

According to one aspect of the present disclosure, there is provided a spraying apparatus that sprays a mist of a sanitizing solution to a subject to sanitize the subject, the spraying apparatus including: a detection unit that detects the subject; a spraying unit provided to spray the mist of the sanitizing solution to a lower body of the subject; and a control unit that controls a spraying operation by the spraying unit on the basis of detection by the detection unit, in which the control unit causes the spraying unit to start spraying the mist in a horizontal direction or a downward direction toward the subject when the detection unit detects the subject.

According to another aspect of the present disclosure, there is provided a spraying apparatus that sprays a mist of a sanitizing solution to a subject to sanitize the subject, the spraying apparatus including: a detection unit that detects the subject; a spraying unit provided to spray the mist of the sanitizing solution to the subject; an illumination apparatus that illuminates a spray range of the sanitizing solution; and a control unit that controls a spraying operation by the spraying unit and an illuminating operation by the illumination apparatus on the basis of detection by the detection unit, in which the control unit causes the spraying unit to start spraying the mist toward the subject and causes the illumination apparatus to start illuminating the spray range of the sanitizing solution when the detection unit detects the subject.

According to yet another aspect of the present disclosure, there is provided a method for operating a spraying apparatus that sprays a mist of a sanitizing solution to a subject to sanitize the subject, the spraying apparatus including a detection unit that detects the subject, a spraying unit provided to spray the mist of the sanitizing solution to a lower body of the subject, and a control unit that controls a spraying operation by the spraying unit on the basis of detection by the detection unit, in which the control unit causes the spraying unit to start spraying the mist in a horizontal direction or a downward direction toward the subject when the detection unit detects the subject, the method including: by the detection unit, detecting the subject; and by the control unit, performing control such that the mist is started to be sprayed in the horizontal direction or the downward direction from the spraying unit toward the subject on the basis of detection of the subject by the detection unit.

According to yet another aspect of the present disclosure, there is provided a method for operating a spraying apparatus for spraying a mist of a sanitizing solution to a subject to sanitize the subject, the spraying apparatus including a detection unit that detects the subject, a spraying unit provided to spray the mist of the sanitizing solution to the subject, an illumination apparatus that illuminates a spray range of the sanitizing solution, and a control unit that controls a spraying operation by the spraying unit and an illumination operation by the illumination apparatus on the basis of detection by the detection unit, in which the control unit causes the spraying unit to start spraying a mist toward the subject and causing the illumination apparatus to start illuminating the spray range of the sanitizing solution when the detection unit detects the subject, the method including: by the detection unit, detecting the subject; and by the control unit, controlling the spraying operation by the spraying unit and the illuminating operation by the illumination apparatus on the basis of detection of the subject by the detection unit.

### Advantageous Effects of Invention

According to the aspects of the present disclosure, since the mist is started to be sprayed from the spraying unit toward the lower body of the subject or the spray range of the mist is illuminated by the illumination apparatus, the mist does not hit the face of the subject, or the subject can easily avoid the illuminated spray range, and the mist can be sprayed while reducing the psychological anxiety of the user, who is the subject.

### BRIEF DESCRIPTION OF DRAWINGS

FIG. 1 is a schematic view of a spraying apparatus according to first and second embodiments of the present disclosure.

FIG. 2A is a side view illustrating a relationship between a spraying unit and an illumination apparatus in FIG. 1.

FIG. 2B is a top view illustrating a relationship between the spraying unit and the illumination apparatus in FIG. 2A,

FIG. 2C is an explanatory view of a third modified embodiment of the present disclosure.

FIG. 2D is an explanatory view of fourth and fifth modified embodiments of the present disclosure.

FIG. 3 is a schematic view of a spraying apparatus according to a modified embodiment of the present disclosure.

FIG. 4A is a side view of another example of the spraying unit according to the modified embodiment of FIG. 3.

FIG. 4B is a top view of the another example of the spraying unit according to the modified embodiment of FIG. 3.

### DESCRIPTION OF EMBODIMENTS

Hereinafter, embodiments of the present disclosure will be described in detail with reference to the drawings.

### First Embodiment

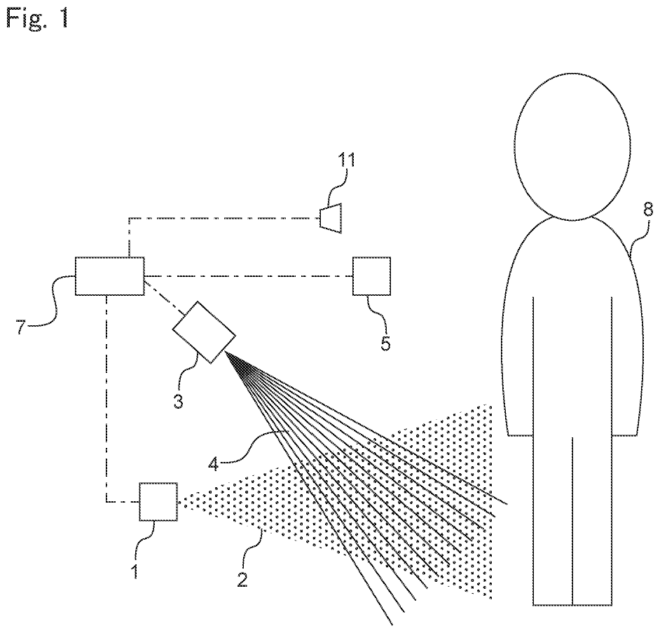

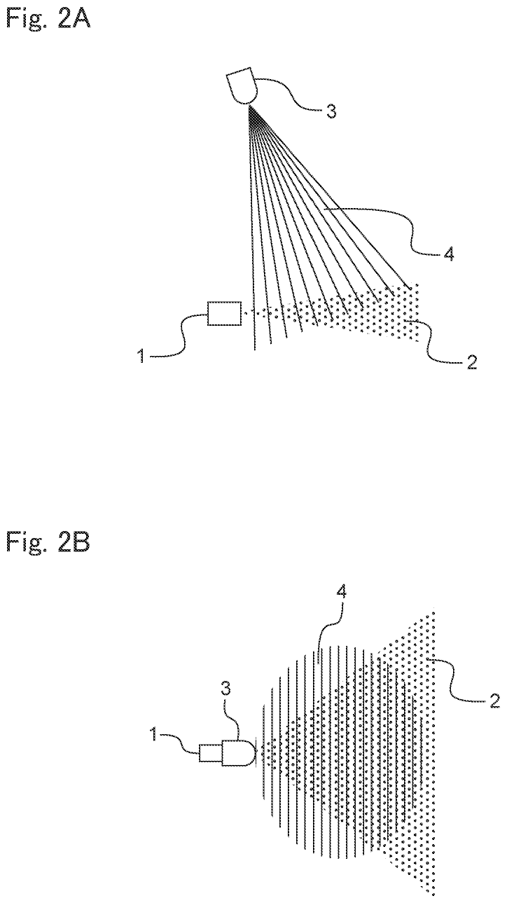

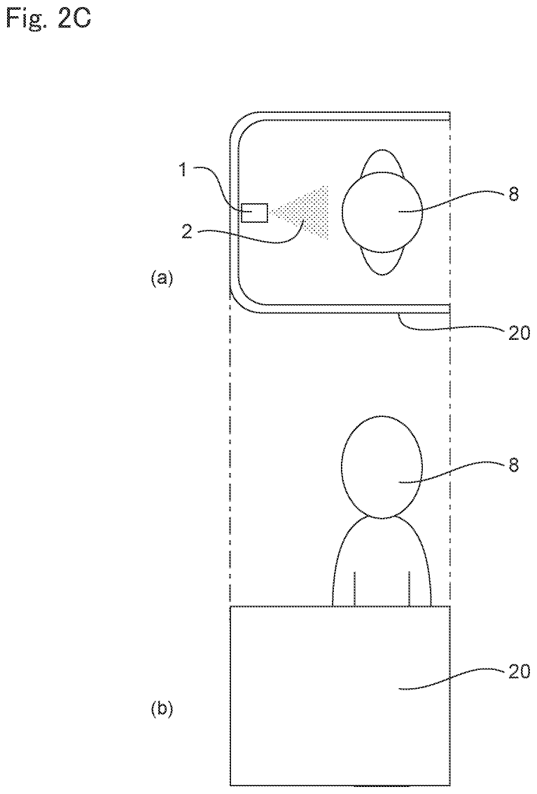

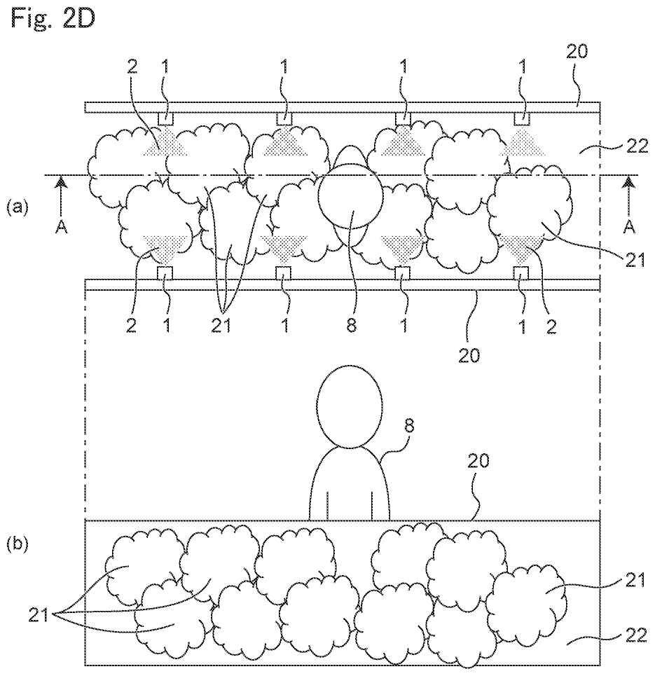

As illustrated in FIGS. 1 to 2B, a spraying apparatus according to a first embodiment of the present disclosure includes a detection unit 5, a spraying unit 1, and a control unit 7, and performs sanitization by spraying a mist of a sanitizing solution to a subject 8. Note that although the subject 8 is actually located within a spray range 2, FIG. 1 illustrates a state where the subject 8 is located outside the spray range 2 for easy understanding.

The detection unit 5 detects the subject 8 such as a person within the spray range 2 of the spraying unit 1. Examples of the detection unit 5 include a lever switch on which a foot steps, a touch sensor, and a non-contact ultrasonic sensor or infrared sensor capable of detecting a subject by placing a hand over the sensor. A detection result of the detection unit 5 is input to the control unit 7.

The spraying unit 1 includes a spray nozzle that sprays water or a sanitizing solution in a mist form to the spray range 2, and is provided so that a spray opening faces the lower body (for example, below the knees) of the subject 8 so as to spray the mist of the sanitizing solution to the lower body of the subject 8 and to the spray range 2. An example of the spray range 2s can be illustrated as a triangular area in the side view of FIG. 2A and a triangular area in the top view of FIG. 2B.

The control unit 7 controls a spraying operation on the basis of detection of the subject 8 by the detection unit 5. For example, when the detection unit 5 detects the subject 8, start of spraying by the spraying unit 1 is controlled, and when the detection unit 5 no longer detects the subject 8, the spraying operation such as stop of spraying is controlled. The stop of spraying may mean automatically stopping spraying after a predetermined time.

When the detection unit 5 detects the subject 8, the control unit 7 starts spraying the mist in a horizontal direction or a downward direction, in other words, toward the lower body of the subject 8, from the spraying unit 1 toward the subject 8.

According to the first embodiment, since the mist is started to be sprayed from the spraying unit 1 toward the lower body of the subject 8, the mist does not hit the face of the subject 8, and it is possible to spray the mist while reducing the psychological anxiety of a user, who is the subject 8.

Note that, as a background of this disclosure, there is a tendency that the value for preventing various bacteria from being brought indoors increases, and various efforts have been made to perform sanitization by spraying a mist to the lower body of a person, who is a hotbed of various bacteria. The present disclosure is an effective approach to this issue.

As still another issue, when an aqueous solution containing hypochlorous acid is used as an example of the sanitizing solution, there is a risk of inflammation in a case where a person directly sucks an unevaporated mist containing hypochlorous acid or in a case where the unevaporated mist containing hypochlorous acid adheres to a sensitive part such as an eye.

Such an issue can be effectively solved in the first embodiment.

Note that as a further additional issue, in a conventional nozzle, there is another issue that a floor is wet in a case where the installation position of the nozzle is set lower than the waist and the mist is sprayed with the spray angle of the mist set to a depression angle with respect to a horizontal plane as the countermeasure.

In order to solve such an additional issue, it is only required to spray a mist having a particle diameter of Sauter mean 7 μm or less as a sanitizing solution, and to install a (for example, two-fluid) nozzle having a spray direction at a depression angle with respect to the horizontal plane at an installation height of 0.7 m or less from the ground surface as a specific example of the spraying unit 1.

As a spraying system, a supply system that supplies an aqueous solution containing hypochlorous acid is connected to the (two-fluid) nozzle. Note that a supply system that supplies compressed air may also be connected to the nozzle.

According to such a configuration, the aqueous solution containing hypochlorous acid is sprayed in mist form into a space after passing through the nozzle.

The mist is ejected from the nozzle adjusted at the depression angle toward the ground surface; however, since the particle diameter is 7 μm or less, the mist completely evaporates or loses volume force by the time the mist reaches the ground surface. Therefore, the mist adheres to the ground and does not cause wetting. As a result, the lower body of a person standing in the spray direction can be effectively sanitized without wetting the ground surface such as a floor.

Note that in a case where a person stands on a grid-like pedestal provided on a spray straight line, the mist goes around to the soles of the feet, and it is possible to effectively sanitize the soles of the feet where a sanitization effect is conventionally weak.

### Second Embodiment

As illustrated in FIGS. 1 to 2B, a spraying apparatus according to a second embodiment of the present disclosure further includes an illumination apparatus 3.

The illumination apparatus 3 includes an LED or the like, and illuminates a spray range 2 of a sanitizing solution by causing an illumination range 4 to overlap the spray range 2 of the sanitizing solution. The illumination apparatus 3 is disposed near a spray opening of a spraying unit. 1, and an illumination direction, that is, an irradiation direction of the illumination apparatus 3 is set to a spray direction of the spraying unit 1 or a direction close to the spray direction. Thus, the spray range 2 and the illumination range 4 are likely to overlap each other. For example, the illumination apparatus 3 can be disposed directly above the spray opening. In FIGS. 1 and 2A, the illumination apparatus 3 is disposed such that a central axis of the spray range 2 and an optical axis of the illumination range 4 intersect at an acute angle.

An example of the spray range 2s can be illustrated as a triangular area in the side view of FIG. 2A and a triangular area in the top view of FIG. 2B, In contrast, an example of the illumination range 4 can be illustrated as a triangular area in the side view of FIG. 2A and an elliptical area in the top view of FIG. 2B. An area where the spray range 2 and the illumination range 4 overlap each other is a spray area visualized by illumination.

Note that the spraying unit 1 of the second embodiment is not limited to the one that sprays the mist in the horizontal direction or the downward direction toward the lower body of the subject 3, and may be one that sprays the mist in an arbitrary direction.

A control unit 7 also controls operation of the illumination apparatus 3. When the spraying unit 1 starts spraying the mist, the control unit 7 starts illumination of the spray range 2 by the illumination apparatus 3. That is, when the sanitizing solution is sprayed from the spraying unit 1, the illumination apparatus 3 emits light toward the mist being sprayed. As a result, the spray range 2 and the illumination range 4 overlap each other, the spray range 2 can be visualized, and the subject 8 can visually recognize the spray range 2. As a result, a person who does not like to be exposed to the mist of the sanitizing solution can avoid the mist by looking at the illumination range 4.

In addition, when the effective range of sanitization is determined by the sanitizing solution, only the effective range can be illuminated to indicate the effective range. Specifically, depending on the type, concentration, spray amount, or the like of the sanitizing solution, the range in which the sanitization effect is effective at the time of spraying the sanitizing solution is not the entire spray range but a limited part thereof. Therefore, by adjusting the illumination range 4 by the control unit 7 on the basis of such information and illuminating the illumination range 4, the effective sanitization range in the spray range 2 can be clearly indicated by the illumination range 4.

Therefore, the control unit 7 may hold or acquire information of the range in which the sanitization effect is effective at the time of spraying the sanitizing solution from the type, concentration, spray amount, or the like of the sanitizing solution, determine the illumination range 4 for the spray range 1 on the basis of the held or acquired information, and illuminate the illumination range 4 for the spray range 1.

For example, the spraying apparatus may include a plurality of types of sanitizing solutions, and the illumination range 4 for the spray range 1 may be made different for each of the sanitizing solutions.

In addition, the color of illumination can be changed depending on the type of a sprayed liquid. For example, when water and a sanitizing solution or two kinds of sanitizing solutions are sprayed, one of the liquids can have a blue illumination range and the other liquid can have a purple illumination range.

### Modified Embodiments

Note that the present disclosure is not limited to the above-described embodiments, and can be implemented in various other aspects.

### First Modified Embodiment

For example, each of the spraying apparatuses may further include a notification unit 11 that is controlled by the control unit 7 and notifies the subject 8 that spraying has stopped.

The control unit 7 controls the spraying unit 1 to stop spraying the sanitizing solution after a predetermined time from the start of spraying, and controls the notification unit 11 to notify the subject 8 that spraying has stopped.

As an example of the notification unit 11, stop of spraying may be notified by sound, light, or a button glowing. Thus, the subject 8 can easily recognize completion of sanitization.

### Second Modified Embodiment

Furthermore, in each of the spraying apparatuses, the detection unit 5 may detect whether or not the subject 8 is stopped, and the control unit 7 may start spraying the mist from the spraying unit 1 to the subject 8 when the detection unit 5 detects that the subject 8 is stopped after the detection unit 5 detects the subject 8.

When it is detected that the subject 8 stops means when the subject 8 steps on the lever switch with the foot, when the subject 8 is in contact with the touch switch for a predetermined time, when the non-contact sensor continues to detect the subject 8 for a predetermined time, and the like.

Efficient sanitization can be performed if the spraying unit 1 starts spraying the mist to the subject 8 when the detection unit 5 detects that the subject 8 is stopped as described above.

### Third Modified Embodiment

In addition, as illustrated in FIG. 20, each of the spraying apparatuses may further include a fence 20 that faces the spray opening of the spraying unit 1 and surrounds the subject 8 from the upper portion to the feet thereof, and may start spraying the mist from the spraying unit 1 to the subject 8 in the spray range 2 in an area surrounded by the fence 20. Note that Figure (a) on the upper side of FIG. 20 is a plan view, and Figure (b) on the lower side of FIG. 20 is a side view (However, for easy understanding, the subject 8 is illustrated by rotating the phase by 90 degrees around the central axis.).

The fence 20 surrounds the subject 8 from the lower part of the upper body to the feet thereof, and includes, for example, a plate member having a U shape in plan view, can block wind and the like, and enables reliable spraying to the subject 8.

With such a configuration, by disposing the fence 20, spraying to the subject 8 can be reliably performed regardless of natural conditions such as wind even outdoors.

### Fourth Modified Embodiment

In addition, as illustrated in FIG. 2D, in each of the spraying apparatuses, for example, a plurality of spraying units 1 may be disposed in a space of a predetermined range such as a passage 22 at intervals along a passage longitudinal direction. The passage 22 is sandwiched by two flat plate-shaped fences 20 and the subject 8 walks therethrough. The plurality of spraying units 1 may spray a mist of the sanitizing solution to form a sea of clouds 21. Note that Figure (a) on the upper side of FIG. 2D is a plan view, and Figure (b) on the lower side of FIG. 2D is a cross-sectional view taken along line A-A of Figure (a) (However, for easy understanding, the subject 8 is illustrated by rotating the phase by 90 degrees around the central axis.). Furthermore, in the sea of clouds 21, the clouds are illustrated independently for easy understanding in the Figure; however, the clouds may be mixed with each other.

With this configuration, the subject 8 can be sanitized only by passing through the sea of clouds 21, and efficient sanitization can be performed.

### Fifth Modified Embodiment

Furthermore, in each of the spraying apparatuses, as illustrated in FIG. 2D, the sea of clouds 21 formed by the spraying units 1 may have a length dimension in consideration of walking time of the subject 8.

With this configuration, the subject 8 can be sufficiently sanitized only by walking in the sea of clouds 21. Therefore, a sanitization effect can be secured by the length of the passage 22 in which the sea of clouds 21 is disposed, and efficient sanitization can be performed.

### Sixth Modified Embodiment

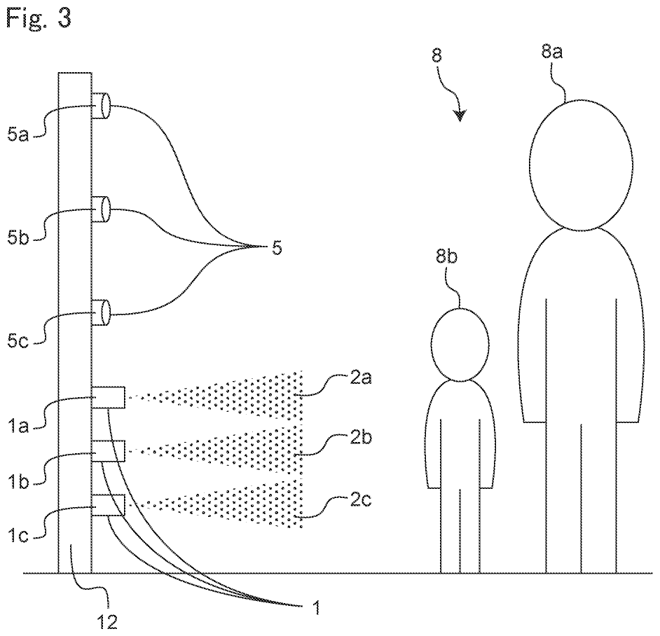

In addition, each of the spraying apparatuses may vary the spray range 2 depending on the height of the subject 8. That is, in each spraying apparatus, as illustrated in FIG. 3, the detection unit 5 may be disposed on the upper side of a fixing plate 12, and the spraying unit 1 may be arranged on the lower side of the fixing plate 12.

The detection unit 5 is configured such that a first detection unit 5a, a second detection unit 5b, and a third detection unit 5c are disposed in this order from top to bottom along the vertical direction at locations having different heights.

The spraying unit 1 is configured such that a first spraying unit 1a, a second spraying unit 1b, and a third spraying unit 1c are disposed in this order from top to bottom along the vertical direction at locations having different heights. The first spraying unit is has a first spray range 2a on the uppermost side and is directed toward a vicinity of an upper portion of the lower body of an adult. The second spraying unit 1b has a second spray range 2b below the first spray range 2a and is directed toward a vicinity of a center portion of the lower body of the adult. The third spraying unit 1c has a third spray range 2c below the second spray range 2b and is directed toward a vicinity of a lower portion of the lower body of the adult or toward the lower body of a child.

A tall person 8a, for example, an adult is detected as the subject 8 in subject detection by both the first detection unit 5a and the second detection unit and the control unit 7 can cause both the first spraying unit 1a and the second spraying unit 1b to start spraying toward the lower body of the adult. Furthermore, a short person 8b, for example, a child is detected as the subject 8 in subject detection by only the third detection unit 5c, and the control unit 7 can cause only the third spraying unit is to start spraying toward the lower body of the child.

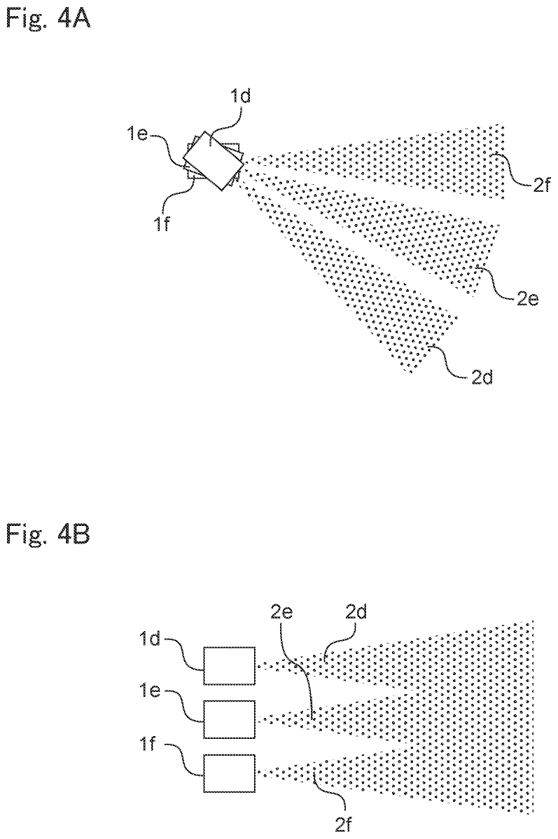

As the spraying unit 1, instead of the configuration in which the three spraying units 1a, 1b, and 1c are disposed with a predetermined distance therebetween along the vertical direction as illustrated in FIG. 3, spraying units may be disposed at the same height in the vertical direction with different spray angles as illustrated in FIGS. 4A and 4B.

That is, the spraying unit 1 is configured such that a fourth spraying unit 1f, a fifth spraying unit 1e, and a sixth spraying unit 1d are disposed at the same position along the vertical direction with spray, angles from upward to downward. The fourth spraying unit 1f has a fourth spray range 2f that is the most upward and is directed toward the vicinity of the upper portion of the lower body of the adult. The fifth spraying unit 1e has a fifth spray range 2e that is more downward than the fourth spray range 2f and is directed toward the vicinity of the center portion of the lower body of the adult. The sixth spraying unit 1d has a sixth spray range 2d. that is more downward than the fifth spray range 2e and is directed toward the vicinity of the lower portion of the lower body of the adult or toward the lower body of the child.

The tall person 8a, for example, an adult is detected as the subject 8 in subject detection by both the first detection unit 5a and the second detection unit 5b, and the control unit 7 can cause both the fourth spraying unit 1f and the fifth spraying unit 1e to start spraying toward the lower body of the adult. Furthermore, the short person 8b, for example, a child is detected as the subject 8 in subject detection by only the third detection unit 5c, and the control unit 7 can cause only the sixth spraying unit 1d to start spraying toward the lower body of the child.

With the configuration as illustrated in FIGS. 3 to 4B, the spray angle can be changed depending on the height of the subject 8, and a psychological load can be reduced even for a short person or a child.

Note that a configuration in which only one spraying unit 1 is disposed, and the spray angle is automatically changed depending on the height to perform spraying is possible (not illustrated).

### Seventh Modified Embodiment

In addition, in each of the spraying apparatuses, the control unit 7 may perform control such that illumination from the illumination apparatus 3 to the spray range 2 of the sanitizing solution is started substantially at the same time as start of spraying, and illumination to the spray range 2 of the sanitizing solution is stopped substantially at the same time as stop of spraying.

With this configuration, injection timing can be Visualized by illumination, and the injection timing can be visually recognized. Therefore, in a case where illumination by the illumination apparatus 3 is earlier than spraying, the mist spray range 2 can be known in advance by the illumination range 4, so the subject 8 can easily avoid the mist spray range. Furthermore, in contrast, in a case where mist spraying is earlier than illumination, the actual mist spray range 2 is accurately visualized by the illumination, which is easy to understand.

Note that by appropriately combining arbitrary embodiments or modified embodiments among the various embodiments or modified embodiments described above, the effects of the respective embodiments or modified embodiments can be achieved. In addition, combinations of embodiments, combinations of working examples, or combinations of an embodiment and a working example are possible, and combinations of features in different embodiments or working examples are also possible.

Although the present disclosure has been fully described in connection with preferred embodiments with reference to the accompanying drawings, various changes and modifications will be apparent to those skilled in the art. Such changes and modifications are to be understood as being included within the scope of the present disclosure as set forth in the appended claims.

### Industrial Applicability

The chemical spraying system and the method for operating the same according to the present disclosure enable spraying while reducing psychological anxiety of the user and are useful for spraying a sanitizing solution as a chemical for sanitization or sterilization.

### REFERENCE SIGNS LIST

1. spraying unit
1a. first spraying unit
1b. second spraying unit
1c. third spraying unit
2. spray range
2a. first spray range
2b. second spray range
2c. third spray range
3. illumination apparatus
4. illumination range
5. detection unit
5a. first detection unit
5b. second detection unit
5c. third detection unit
7. control unit
8. subject
8a. tall person
8b. short person
11. notification unit
12. fixing plate
20. fence
21. sea of clouds
22. passage

1. A spraying apparatus that sprays a mist of a sanitizing solution to a subject to sanitize the subject, the spraying apparatus comprising:
a detection unit that detects the subject;
a spraying unit provided to spray the mist of the sanitizing solution to a lower body of the subject; and
a control unit that controls a spraying operation by the spraying unit on a basis of detection by the detection unit,
wherein the control unit causes the spraying unit to start spraying the mist in a horizontal direction or a downward direction toward the subject when the detection unit detects the subject.

2. The spraying apparatus according to claim 1 further comprising an illumination apparatus that illuminates a spray range of the sanitizing solution,
wherein
the control unit also controls operation of the illumination apparatus, and
the control unit causes the illumination apparatus to start illumination of the spray range when the spraying unit starts spraying the mist.

3. A spraying apparatus that sprays a mist of a sanitizing solution to a subject to sanitize the subject, the spraying apparatus comprising:
a detection unit that detects the subject;
a spraying unit provided to spray the mist of the sanitizing solution to the subject;
an illumination apparatus that illuminates a spray range of the sanitizing solution; and
a control unit that controls a spraying operation by the spraying unit and an illuminating operation by the illumination apparatus on a basis of detection by the detection unit,
wherein the control unit causes the spraying unit to start spraying the mist toward the subject and causes the illumination apparatus to start illuminating the spray range of the sanitizing solution when the detection unit detects the subject.

4. The spraying apparatus according to claim 1, further comprising a notification unit that is controlled by the control unit and notifies the subject that the spraying has stopped,
wherein the control unit controls the spraying unit to stop spraying the sanitizing solution after a predetermined time from start of the spraying, and controls the notification unit to notify the subject that the spraying has been stopped.

5. The spraying apparatus according to claim 1, wherein
the detection unit detects whether or not the subject is topped, and
the control unit causes the spraying unit to starts spraying the mist to the subject when the detection unit detects the subject and detects that the subject is stopped.

6. The spraying apparatus according to claim 1, further comprising a fence that faces a spray opening of the spraying unit and surrounds the subject from a lower portion of an upper body to feet of the subject,
wherein the mist is started to be sprayed from the spraying unit to the subject in an area surrounded by the fence.

7. The spraying apparatus according to claim 1, wherein the spraying unit sprays the mist of the sanitizing solution to a space in a predetermined range to form a sea of clouds.

8. The spraying apparatus according to claim 7, wherein the sea of clouds formed by the spraying unit has a length dimension in consideration of walking time of the subject.

9. The spraying apparatus according to claim 1, wherein
the detection unit is configured such that a first detection unit, a second detection unit, and a third detection unit are disposed in this order from top to bottom along a vertical direction,
the spraying unit is configured such that a first spraying unit, a second spraying unit, and a third spraying unit are disposed in this order from top to bottom along the vertical direction, and
the control unit
detects a tall person as the subject in subject detection by both the first detection unit and the second detection unit, and causes both the first spraying unit and the second spraying unit to start the spraying, and
detects a short person as the subject in subject detection only by the third detection unit, and causes only the third spraying unit to start the spraying.

10. The spraying apparatus according to claim 2, wherein the control unit performs control such that
the illumination from the illumination apparatus to the spray range of the sanitizing solution is started substantially at a same time as start of the spraying, and
the illumination to the spray range of the sanitizing solution is stopped substantially at a same time as stop of the spraying.

11. A method for operating a spraying apparatus that sprays a mist of a sanitizing solution to a subject to sanitize the subject, the spraying apparatus including
a detection unit that detects the subject,
a spraying unit provided to spray the mist of the sanitizing solution to a lower body of the subject, and
a control unit that controls a spraying operation by the spraying unit on a basis of detection by the detection unit,
the control unit causing the spraying unit to start spraying the mist in a horizontal direction or a downward direction toward the subject when the detection unit detects the subject,
the method comprising:
by the detection unit, detecting the subject; and
by the control unit, performing control such that the mist is started to be sprayed in the horizontal direction or the downward direction from the spraying unit toward the subject on a basis of detection of the subject by the detection unit.

12. The method for operating the spraying apparatus according to claim 11,
wherein the spraying apparatus further includes an illumination apparatus that illuminates a spray range of the sanitizing solution,
the control unit also controls operation of the illumination apparatus, and
the control unit performs control such that the illumination apparatus starts illuminating the spray range when the spraying unit starts spraying the mist.

13. A method for operating a spraying apparatus that sprays a mist of a sanitizing solution to a subject to sanitize the subject, the spraying apparatus including
a detection unit that detects the subject,
a spraying unit provided to spray the mist of the sanitizing solution to the subject,
an illumination apparatus that illuminates a spray range of the sanitizing solution, and
a control unit that controls a spraying operation by the spraying unit and an illumination operation by the illumination apparatus on a basis of detection by the detection unit,
the control unit causing the spraying unit to start spraying the mist toward the subject and causing the illumination apparatus to start illuminating the spray range of the sanitizing solution when the detection unit detects the subject,
the method comprising:
by the detection unit, detecting the subject; and
by the control unit, controlling the spraying operation by the spraying unit and the illuminating operation by the illumination apparatus on a basis of detection of the subject by the detection unit.

14. The spraying apparatus according to claim 2 further comprising a notification unit that is controlled by the control unit and notifies the subject that the spraying has stopped,
wherein the control unit controls the spraying unit to stop spraying the sanitizing solution after a predetermined time from start of the spraying, and controls the notification unit to notify the subject that the spraying has been stopped.

15. The spraying apparatus according to claim 2, wherein
the detection unit detects whether or not the subject is topped, and
the control unit causes the spraying unit to starts spraying the mist to the subject when the detection unit detects the subject and detects that the subject is stopped.

16. The spraying apparatus according to claim 2 further comprising a fence that faces a spray opening of the spraying unit and surrounds the subject from a lower portion of an upper body to feet of the subject,
wherein the mist is started to be sprayed from the spraying unit to the subject in an area surrounded by the fence.

17. The spraying apparatus according to claim 2, wherein the spraying unit sprays the mist of the sanitizing solution to a space in a predetermined range to form a sea of clouds.

18. The spraying apparatus according to claim 2, wherein
the detection unit is configured such that a first detection unit, a second detection unit, and a third detection unit are disposed in this order from top to bottom along a vertical direction,
the spraying unit is configured such that a first spraying unit, a second spraying unit, and a third spraying unit are disposed in this order from top to bottom along the vertical direction, and
the control unit
detects a tall person as the subject in subject detection by both the first detection unit and the second detection unit, and causes both the first spraying unit and the second spraying unit to start the spraying, and
detects a short person as the subject in subject detection only by the third detection unit, and causes only the third spraying unit to start the spraying.

19. The spraying apparatus according to claim 3, wherein the control unit performs control such that
the illumination from the illumination apparatus to the spray range of the sanitizing solution is started substantially at a same time as start of the spraying, and
the illumination to the spray range of the sanitizing solution is stopped substantially at a same time as stop of the spraying.
